(19) United States
(12) Patent Application Publication
(10) Pub. No.: US 2022/0009951 A1
(43) Pub. Date: Jan. 13, 2022

(54) TORSEMIDE PHOSPHATE PRODRUG, PREPARATION METHOD AND COMPOSITION THEREOF

(71) Applicant: Shanghai Xunhe Pharmaceutical Technology Co. Ltd., Shanghai (CN)

(72) Inventors: Yongyong ZHENG, Shanghai (CN); Nongnong WEI, Shanghai (CN); Hua JIN, Shanghai (CN); Feng ZHOU, Shanghai (CN); Meihua HUANG, Shanghai (CN)

(21) Appl. No.: 17/482,827

(22) Filed: Sep. 23, 2021

Related U.S. Application Data
(63) Continuation of application No. PCT/CN2020/084803, filed on Apr. 14, 2020.

(30) Foreign Application Priority Data
May 23, 2019 (CN) 201910435995.7

(51) Int. Cl. C07F 9/58

(52) U.S. Cl. CPC C07F 9/58

(57) ABSTRACT

The present application relates to the technical field of biological medicine, in particular to a torsemide phosphate prodrug, a preparation method and a composition thereof. The torsemide prodrug N-hydroxymethyl-torsemide phosphate, and/or a pharmaceutical salt thereof provided by the present application have a better solubility than torsemide, and have the advantage of high druggability.

### CROSS-REFERENCE TO RELATED APPLICATIONS

The present application is a continuation application of PCT application No. PCT/CN2020/084803, filed on Apr. 14, 2020, which claims the priority of China patent application number CN 2019104359957, filed on May 23, 2019, both of which are hereby incorporated by reference in their entireties.

### TECHNICAL FIELD

The present application relates to the technical field of biological medicine, in particular to a torsemide phosphate prodrug, a preparation method and a composition thereof.

### BACKGROUND

Torsemide has the chemical name 1-[4-(3-methylphenyl)aminopyridin-3-yl]sulfonyl-3-isopropylurea and is a new generation of high-efficiency loop diuretic with a pKa value of 6.44. Torsemide is almost insoluble in water and slightly soluble in 0.1 mol/L sodium hydroxide solution. More than 20 years of clinical application has proved that torsemide has a wide range of indications, and rapid, powerful and long-lasting diuretic effects, which is a class of high-efficiency diuretic that is worth promoting clinically.

At present, torsemide is marketed as injections, tablets, and capsules. In the preparation process of injections, it is hoped that the drug substance has high water solubility. Torsemide is very slightly soluble in water (European Journal of Pharmaceutics and Biopharmaceutics 53 (2002) 75-86). When preparing torsemide injection, sodium hydroxide and a large amount of excipients need to be added to aid dissolution. The excipients used include: polyethylene glycol 400, tromethamine, sodium hydroxide, and hydrochloric acid. The addition of the above-mentioned excipients brings many disadvantages: 1) the process of dissolving torsemide in the sodium hydroxide aqueous solution is significantly exothermic, which is easy to produce degradation impurities of preparation; 2) the addition of organic cosolvents such as polyethylene glycol 400 and tromethamine brings hidden dangers to the safety of injections. People always expect to reduce the number of ingredients in the formula in order to reduce the possible side effects on the patient.

Therefore, it has become a big challenge to develop a new type of loop diuretic with higher water solubility which is more conducive to prepare a preparation.

The present application provides a compound of torsemide phosphate of formula I or a pharmaceutical salt thereof.

The torsemide phosphate prodrug of the present application has the characteristics of high solubility and stability, making it convenient to prepare a preparation, and so on, which is easily industrialized and used for medical purposes.

### SUMMARY

The object of the present application is to provide a torsemide phosphate prodrug of formula I, and a pharmaceutical salt thereof:

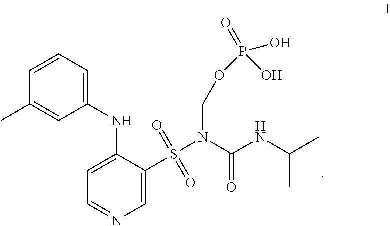 [SMILES: Cc1cccc(Nc2ccncc2S(=O)(=O)N(COP(=O)(O)O)C(=O)NC(C)C)c1]

The chemical name of the above formula is N-hydroxymethyl-torsemide phosphate.

Preferably, the pharmaceutical salt of torsemide phosphate prodrug comprises a pharmaceutically acceptable salt, which is selected from a sodium salt, a potassium salt, a barium salt, a magnesium salt, a zinc salt, a lithium salt, a ferric salt, a ferrous salt or an organic ammonium salt.

Further preferably, the pharmaceutical salt of the torsemide phosphate prodrug is selected from disodium salt, dipotassium salt or organic ammonium salt of phosphate group.

Further, the organic ammonium salt is selected from trimethylammonium salt, triethylammonium salt, tripropylammonium salt, or tri-n-butylammonium salt.

As a preferred embodiment of the present application, the N-hydroxymethyl-torsemide phosphate prodrug is selected from the following compounds:

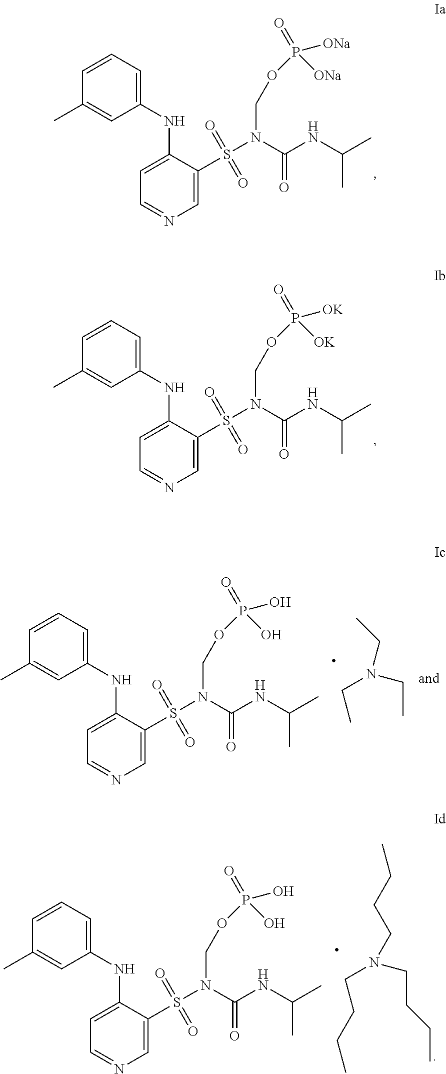

A second object of the present application is to provide a method for preparing the above-mentioned torsemide phosphate prodrug, comprising the following steps:
(1) performing a hydroxymethylation reaction of the torsemide of formula 1 with paraformaldehyde to produce a compound of formula 2;
(2) making the compound of formula 2 undergo chlorination, esterification and debenzylation via hydrogenation to produce the compound of formula I;

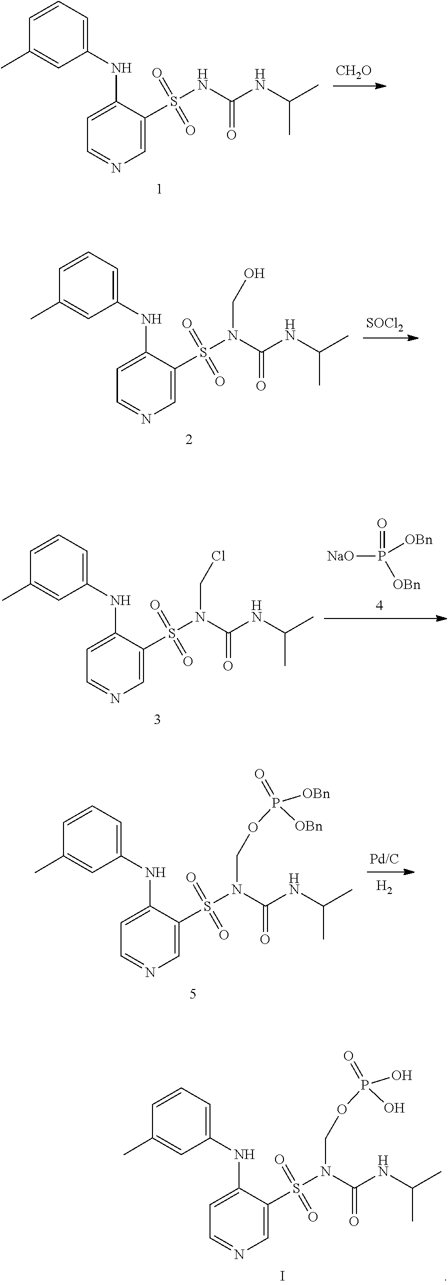

The present application also provides a method for preparing a pharmaceutical salt based on the above-mentioned torsemide phosphate prodrug. The compound of formula I is reacted with sodium hydroxide, potassium hydroxide, triethylamine or tri-n-butylamine respectively, to produce the compound of formula Ia, Ib, Ic, Id:

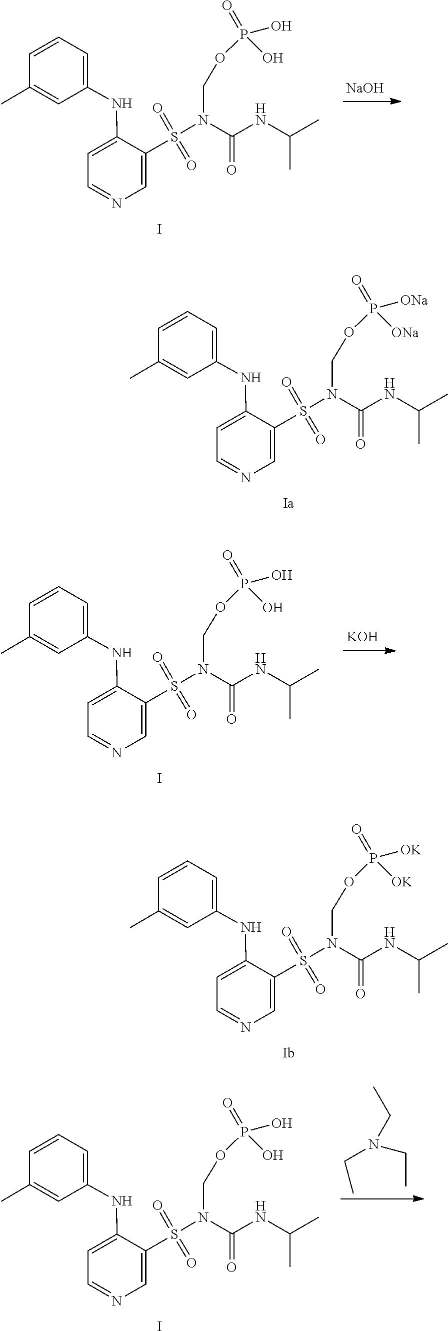

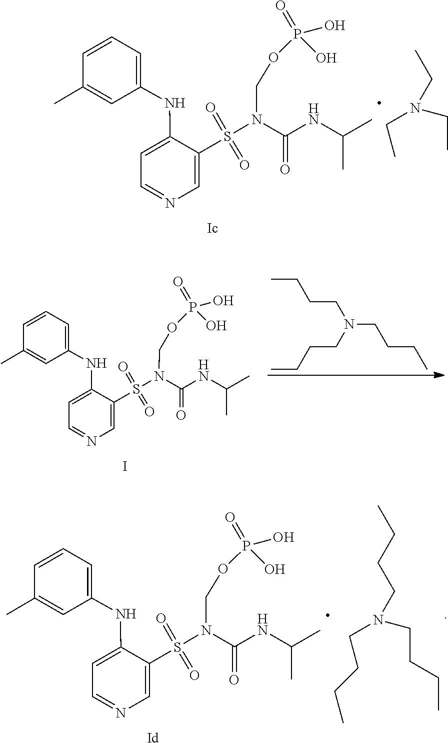

A third object of the present application is to provide a pharmaceutical composition, including a therapeutic amount of N-hydroxymethyl-torsemide phosphate, and/or a pharmaceutical salt thereof, and a pharmaceutically acceptable excipient.

The beneficial effect of the present application is to provide a torsemide prodrug N-hydroxymethyl-torsemide phosphate, and/or a pharmaceutical salt thereof, which have a better solubility than torsemide, and have the advantage of high druggability.

### DETAILED DESCRIPTION OF THE APPLICATION

In the following, the present application will be further described in detail with reference to the embodiments, but it is not limited thereto.

### Example 1 Preparation of N-Hydroxymethyl-Torsemide Phosphate (I)

### Step 1: Preparation of N-hydroxymethyl-torsemide (2)

In a 500 mL reaction flask, were placed anhydrous ethanol (300 mL), torsemide (34.8 g, 0.1 mol, 1 eq) and sodium carbonate (15.9 g, 0.15 mol, 1.5 eq). Paraformaldehyde (15 g, 0.5 mol, 5 eq) was added in batches while stirring. After the addition, the reaction mixture was heated to 80-85° C. to react for 2 hours and cooled slowly to 20-25° C., and a white solid was precipitated, filtered and washed with water. The filter cake was vacuum dried (40° C.) to give N-hydroxymethyl-torsemide (2) (32.5 g, 86% yield), MS: 379 [M+1].

### Step 2: Preparation of N-Chloromethyl-Torsemide (3)

In a 500 mL reaction flask, were placed dichloromethane (200 mL), N,N-dimethylformamide (2 mL) and compound of formula 2 (30 g, 79.3 mmol, 1 eq). Thionyl chloride (28.3 g, 237.8 mmol, 3 eq) was added dropwise while stirring. After the addition, the reaction mixture was heated to 60-65° C. to react for 2 hours. After the reaction was completed, the reaction mixture was poured into a 500 mL beaker, and 10% sodium carbonate aqueous solution (50 mL) was added dropwise in batches under an ice bath. The water layer was separated by a separatory funnel. The organic layer was washed twice with water (50 mL×2) and once with saturated brine (50 mL), separated, dried over anhydrous sodium sulfate, and concentrated. The residue was beating washed with ethyl acetate (50 mL) and filtered, and the filter cake was vacuum dried (40° C.) to give N-chloromethyl-torsemide (3) (29.9 g, 95% yield), MS: 398 [M+1].

### Step 3: Preparation of N-Hydroxymethyl-Torsemide Dibenzyl Phosphate (5)

In a 500 mL reaction flask, were placed acetonitrile (300 mL), compound of formula 3 (29 g, 73.1 mmol, 1 eq), sodium carbonate (15.5 g, 146.1 mmol, 2 eq), dibenzyl phosphate sodium salt of formula 4 (24.1 g, 80.4 mmol, 1.1 eq). The reaction mixture was heated to 80-85° C. to react for 8 hours while stirring, and filtered to remove the inorganic salts while it is hot. The filtrate was concentrated and the concentrated residue was recrystallized with toluene (50 mL) and filtered. The filter cake was vacuum dried (50° C.) to give N-hydroxymethyl-torsemide dibenzyl phosphate (5) (21.5 g, 46% yield), MS: 639 [M+1].

### Step 4: Preparation of N-Hydroxymethyl-Torsemide Phosphate (I)

In an autoclave, were placed anhydrous ethanol (400 mL), compound of formula 5 (20 g, 31.3 mmol, 1 eq), 10% palladium on carbon (2 g, 10% by weight). The gas in the autoclave was replaced with nitrogen three times, and hydrogen was introduced to get a pressure of 2 MPa. The mixture was reacted at room temperature for 5 hours while stirring. After the reaction was completed, the mixture was filtered, and the filtrate was concentrated to give N-hydroxymethyl-torsemide phosphate (I) (11.5 g, 80% yield) as a white solid, MS: 459 [M+1]. 1H NMR (400 MHz, D2O) δ: 8.56 (s, 1H), 7.99-8.00 (d, J=4.0 Hz, 1H), 7.23-7.26 (m, 1H), 6.98-7.03 (m, 3H), 6.89-6.90 (m, 1H), 5.91 (s, 2H), 3.55-3.57 (m, 1H), 2.25 (s, 3H), 0.95 (s, 3H), 0.94 (s, 3H).

### Example 2 Preparation of Disodium N-Hydroxymethyl-Torsemide Phosphate (Ia)

In a 100 mL reaction flask, were placed anhydrous ethanol (50 mL) and N-hydroxymethyl-torsemide phosphate (I) (10 g, 21.8 mmol, 1 eq), and 25% sodium hydroxide solution (1.83 g, 45.8 mmol, 2.1 eq) was added dropwise while stirring. After the addition, the mixture was stirred for 1 h. The reaction solution was added with acetone (50 mL), stirred for 30 min and filtered to give the crude disodium salt. The obtained crude product was added to acetone (50 mL)/H2O (5 mL) system, recrystallized, and filtered. The filter cake was vacuum dried (50° C.) to give disodium N-hydroxymethyl-torsemide phosphate (Ia) (7.6 g, yield 69%) with a purity of 99.90% by HPLC. MS: 503 [M+1], 1H NMR (400 MHz, D2O) δ: 8.55 (s, 1H), 7.99 (d, J=4.0 Hz, 1H), 7.25 (m, 1H), 6.98-7.05 (m, 3H), 6.92 (m, 1H), 5.92 (s, 2H), 3.56 (m, 1H), 2.24 (s, 3H), 0.94 (s, 3H), 0.93 (s, 3H). Sodium content: 9.19%.

### Example 3 Preparation of Dipotassium N-Hydroxymethyl Torsemide Phosphate (Ib)

In a 100 mL reaction flask, were placed anhydrous ethanol (50 mL) and N-hydroxymethyl-torsemide phosphate (I) (10 g, 21.8 mmol, 1 eq), and 20% potassium hydroxide solution (2.57 g, 45.8 mmol, 2.1 eq) was added dropwise while stirring. After the addition, the mixture was stirred for 1 h. The reaction solution was added with acetone (50 mL), stirred for 30 min and filtered to obtain the crude dipotassium salt. The obtained crude product was added to acetone (50 mL)/H2O (5 mL) system, recrystallized, and filtered. The filter cake was vacuum dried (50° C.) to give dipotassium N-hydroxymethyl-torsemide phosphate (Ib) (7.6 g, yield 65%) with a purity of 99.92% by HPLC. MS: 535 [M+1], 1H NMR (400 MHz, D2O) δ: 8.57 (s, 1H), 7.99 (d, J=4.0 Hz, 1H), 7.27 (m, 1H), 6.99-7.04 (m, 3H), 6.94 (m, 1H), 5.91 (s, 2H), 3.55 (m, 1H), 2.23 (s, 3H), 0.94 (s, 6H). Potassium content: 14.58%.

### Example 4 Preparation of N-Hydroxymethyl-Torsemide Phosphate Triethylammonium Salt (Ic)

In a 100 mL reaction flask, were placed anhydrous ethanol (50 mL), N-hydroxymethyl-torsemide phosphate (I) (10 g, 21.8 mmol, 1 eq) and triethylamine (2.2 g, 21.8 mmol, 1 eq), which were stirred for 1 h. The mixture was concentrated to give a foamy solid, which was recrystallized with acetone (30 mL), and filtered. The filter cake was vacuum dried (40° C.) to give N-hydroxymethyl-torsemide phosphate triethylammonium salt (Ic) (6.7 g, yield 55%) with a purity of 99.85% by HPLC. MS: 459 [M+1], 1H NMR (400 MHz, D2O) δ: 8.55 (s, 1H), 7.99 (d, J=4.0 Hz, 1H), 7.25 (m, 1H), 6.97-7.04 (m, 3H), 6.95 (m, 1H), 5.91 (s, 2H), 3.57 (m, 1H), 3.07 (m, 6H), 2.25 (s, 3H), 1.07 (m, 9H), 0.93 (s, 6H).

### Example 5 Preparation of N-Hydroxymethyl-Torsemide Phosphate Tri-n-Butylammonium Salt (Id)

In a 100 mL reaction flask, were placed anhydrous ethanol (50 mL), N-hydroxymethyl-torsemide phosphate (I) (10 g, 21.8 mmol, 1 eq) and tri-n-butylamine (4.04 g, 21.8 mmol, 1 eq), which were stirred for 1 h. The mixture was concentrated to give a foamy solid, which was recrystallized with acetone (30 mL), and filtered. The filter cake was vacuum dried (40° C.) to give N-hydroxymethyl-torsemide phosphate tri-n-butylammonium salt (Id) (7.2 g, yield 51%) with a purity of 99.88% by HPLC. MS: 459 [M+1], 1H NMR (400 MHz, D2O) δ: 8.56 (s, 1H), 8.01-8.02 (d, J=4.0 Hz, 1H), 7.26 (m, 1H), 6.98-7.04 (m, 3H), 6.94 (m, 1H), 5.93 (s, 2H), 3.55 (m, 1H), 3.05 (m, 6H), 2.23 (s, 3H), 1.35-1.42 (m, 12H), 0.93 (s, 6H), 0.87 (m, 9H).

### Example 6 Preparation of Disodium N-Hydroxymethyl Torsemide Phosphate Injection

Composition: 10 g disodium N-hydroxymethyl torsemide phosphate, 2000 mL water for injection.

Preparation:

(1) 10 g disodium N-hydroxymethyl-torsemide phosphate was added to 2000 mL water for injection, stirred evenly, and pre-filtered through a plate and frame filter to obtain solution A;
(2) the solution A in step (1) was sterilized and filtered with two 0.22 μm polyethersulfone filter elements to obtain an intermediate B;
(3) the intermediate B was filled, melt-sealed, and packaged to obtain the product.

### Example 7 Preparation of Disodium N-Hydroxymethyl Torsemide Phosphate Lyophilized Powder Injection

Composition: 10 g disodium N-hydroxymethyl-torsemide phosphate, 2000 mL water for injection.

Preparation is as follows:

(1) the selected weight of disodium N-hydroxymethyl-torsemide phosphate was added to 70% of the selected volume of water for injection, stirred until complete dissolution, to obtain solution A;
(2) 30% of the selected volume of water for injection was added to the above solution A. The pH value was adjusted to 8.5 to 9.5 under stirring. The solution was pre-filtered through a plate and frame filter to obtain solution B;
(3) the solution B in step (2) was sterilized and filtered with two 0.22 μm polyethersulfone filter elements to obtain solution C, which was filled and half plugged to obtain intermediate D;
(4) the intermediate D was subjected to freeze-drying treatment at a temperature of −40° C. to −50° C. and a pressure of 10 Pa to 22 Pa, and the freeze-drying treatment adopts the following procedure to raise the temperature:
(a) set the temperature to −45° C.˜−30° C., to pre-freeze for 2.0 h;
(b) raise the temperature to −30° C.˜−20, to sublimate for 4.0 h;
(c) raise the temperature to −20° C.˜−10° C., to sublimate for 1.5 h;
(d) raise the temperature to −10° C.˜0° C., to sublimate for 1.0 h;
(e) raise the temperature to 0° C.˜15° C., to sublimate for 1.5 h;
(f) raise the temperature to 15° C.˜25° C., and keep the temperature for 2.0 h;
the disodium N-hydroxymethyl-torsemide phosphate lyophilized powder injection was obtained by stoppering, taking out of the box and capping.

Solubility Comparison:

Comparing the solubility of Torsemide, N-hydroxymethyl-torsemide phosphate (I), disodium N-hydroxymethyl torsemide phosphate (Ia), dipotassium N-hydroxymethyl-torsemide phosphate (Ib), N-hydroxymethyl-torsemide phosphate triethylammonium salt (Ic), N-hydroxymethyl-torsemide phosphate tri-n-butylammonium salt (Id), the results are as follows:

TABLE 1
Comparison of water solubility of different forms of samples

| Sample | API: Water | | Phenomenon | Conclusion |
|---|---|---|---|---|
| Torsemide | 3 mg: | 10 mL | completely soluble | very slightly |
| | | | | soluble |
| Compound of formula | 0.5 g: | 10 mL | completely soluble | soluble |
| I (Example 1) | | | | |
| Compound of formula | 2 g: | 10 mL | completely soluble | easily soluble |
| Ia (Example 2) | | | | |
| Compound of formula | 1.8 g: | 10 mL | completely soluble | easily soluble |
| Ib (Example 3) | | | | |
| Compound of formula | 1.2 g: | 10 mL | completely soluble | easily soluble |
| Ic (Example 4) | | | | |
| Compound of formula | 1 g: | 10 mL | completely soluble | easily soluble |
| Id (Example 5) | | | | |

The result of solubility test shows that the solubility of the N-hydroxymethyl-torsemide phosphate prodrug in Examples 1 to 5 is better than that of torsemide, and it has the advantage of high druggability.

Comparison of Diuretic Effects:

Male SD rats (body weight 180±20 g) were divided into seven groups randomly, three rats a group, and each rat was administered intragastrically 30 mL/kg normal saline. After intragastric administration of normal saline, except the blank control group, each group was given one drug (10 mg/kg, iv, 1 mg/mL, formulation prescription: 0.5% methylcellulose), and the urination was collected for 4 hours. The results are shown in Table 2:

TABLE 2
Comparison of urine output of different compounds

| | Dosage and method of | Urine output |
|---|---|---|
| Compound | administration | (mL/kg, 4 h) |
| Blank control | — | 18.9 |
| Torasemide | 10 mg/kg, iv | 100.3 |
| Compound of formula I | 10 mg/kg, iv | 105.4 |
| Compound of formula Ia | 10 mg/kg, iv | 121.6 |
| Compound of formula Ib | 10 mg/kg, iv | 116.8 |
| Compound of formula Ic | 10 mg/kg, iv | 103.4 |
| Compound of formula Id | 10 mg/kg, iv | 105.6 |

The result of diuretic effect test shows that the N-hydroxymethyl-torsemide phosphate prodrug in Examples 1 to 5 has a diuretic effect similar to torsemide or better than torsemide, which has the advantage of high druggability.

All documents mentioned in the present application are cited as references in this application, just as if each document is individually cited as a reference.

What is claimed is:

1. A torsemide phosphate prodrug of formula I, or a pharmaceutical salt thereof,

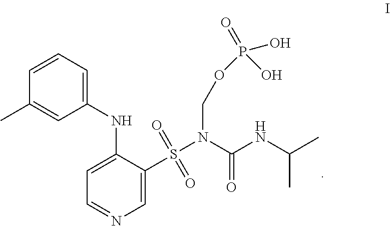 [SMILES: Cc1cccc(Nc2ccncc2S(=O)(=O)N(COP(=O)(O)O)C(=O)NC(C)C)c1]

2. The torsemide phosphate prodrug or pharmaceutical salt thereof according to claim 1, wherein the pharmaceutical salt of the torsemide phosphate prodrug comprises a pharmaceutically acceptable salt, which is selected from a sodium salt, a potassium salt, a barium salt, a magnesium salt, a zinc salt, a lithium salt, a ferric salt, a ferrous salt or an organic ammonium salt.

3. The torsemide phosphate prodrug or pharmaceutical salt thereof according to claim 2, wherein the pharmaceutical salt of the torsemide phosphate prodrug is selected from disodium salt, dipotassium salt or organic ammonium salt of phosphate group.

4. The torsemide phosphate prodrug or pharmaceutical salt thereof according to claim 3, wherein the organic ammonium salt is selected from trimethylammonium salt, triethylammonium salt, tripropylammonium salt, or tri-n-butylammonium salt.

5. The torsemide phosphate prodrug or pharmaceutical salt thereof according to claim 1, wherein the pharmaceutical salt of the torsemide phosphate prodrug is selected from the following compounds:

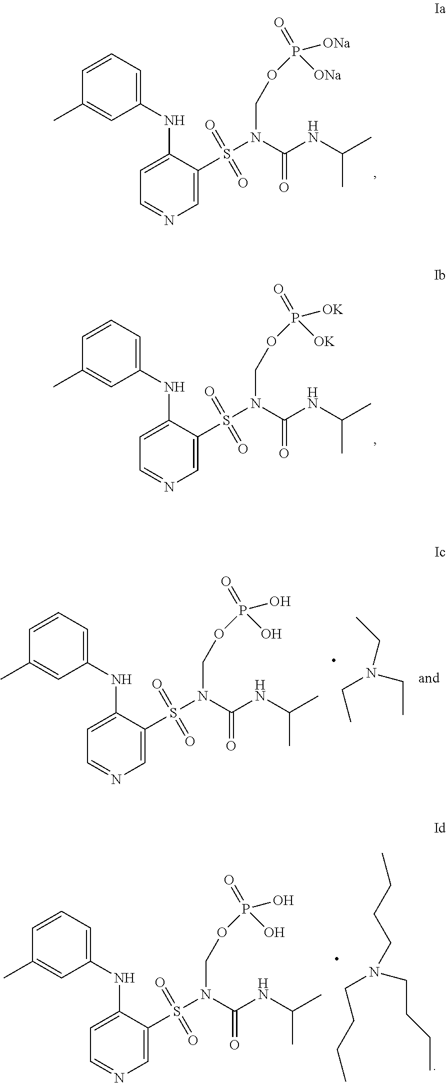

6. A method for preparing the torsemide phosphate prodrug according to claim 1, comprising the following steps:
(1) performing a hydroxymethylation reaction of the torsemide of formula 1 with paraformaldehyde to produce a compound of formula 2;
(2) making the compound of formula 2 undergo chlorination, esterification and debenzylation via hydrogenation to produce the compound of formula I;

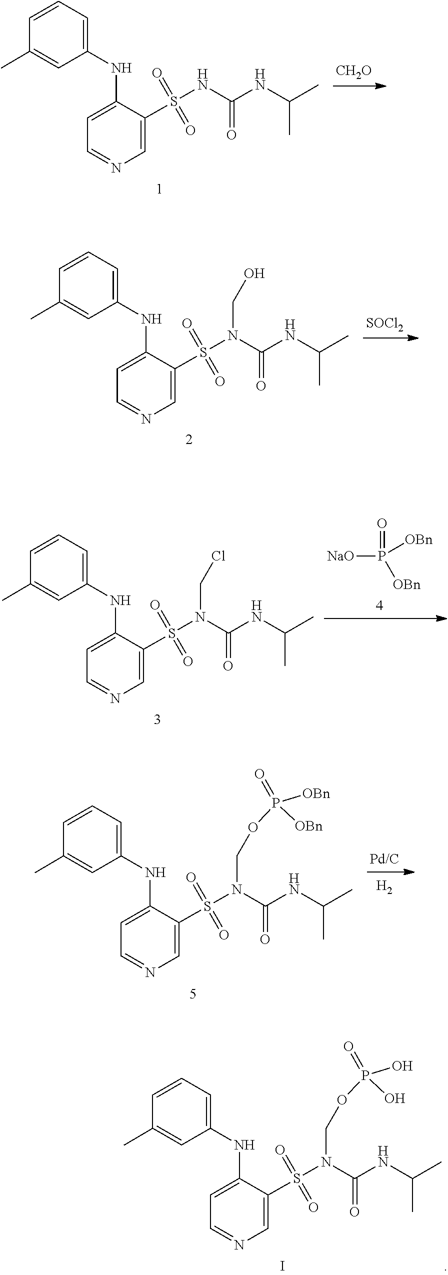

7. The method according to claim 6, further comprising the following steps:
reacting the compound of formula I with sodium hydroxide, potassium hydroxide, triethylamine or tri-n-butylamine respectively, to produce the compound of formula Ia, Ib, Ic, Id respectively:

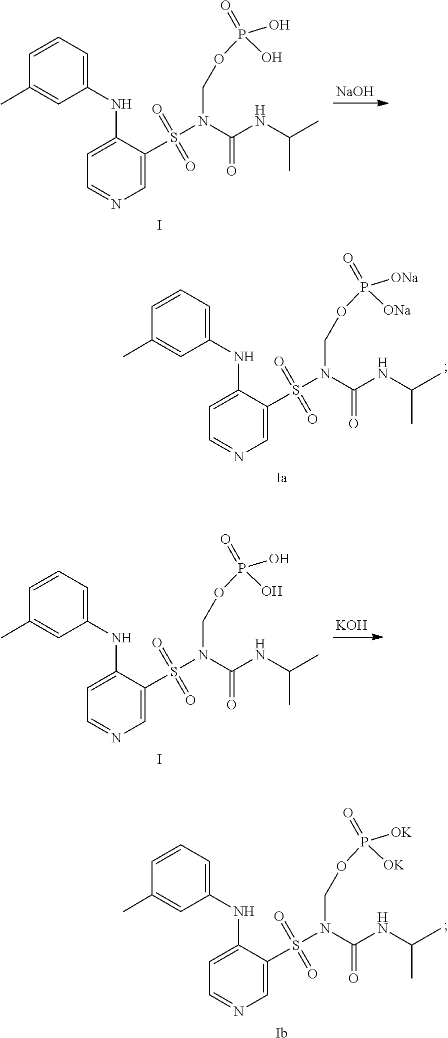

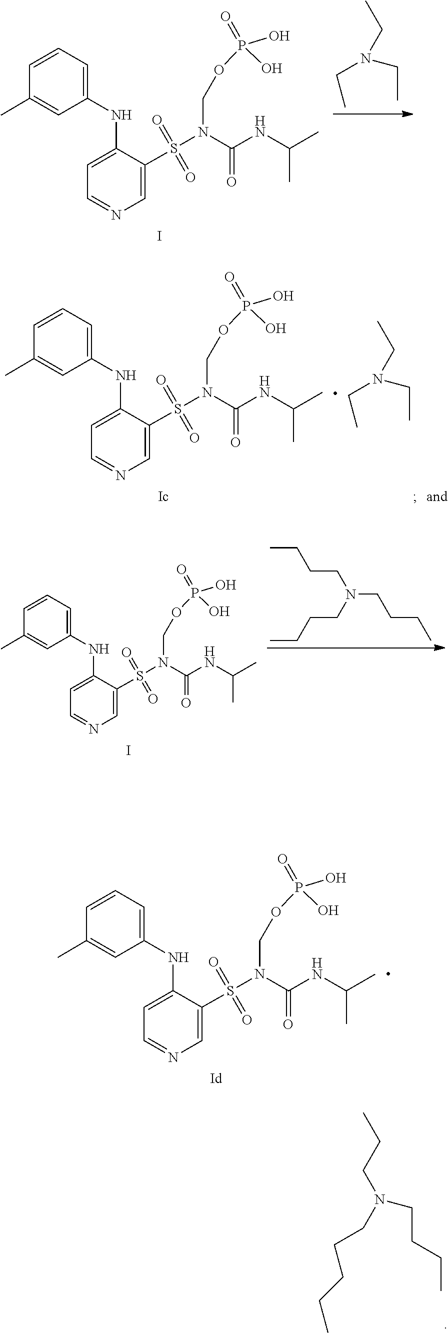

8. A pharmaceutical composition, comprising a therapeutic amount of N-hydroxymethyl-torsemide phosphate and/or pharmaceutical salt thereof according to claim 1, and a pharmaceutically acceptable excipient.

9. A pharmaceutical composition, comprising a therapeutic amount of N-hydroxymethyl-torsemide phosphate, and/or pharmaceutical salt according to claim 5, and a pharmaceutically acceptable excipient.
